(19) United States
(12) Patent Application Publication
(10) Pub. No.: US 2022/0249397 A1
(43) Pub. Date: Aug. 11, 2022

(54) Automated Dosing Protocol for Use of Cannabinoids or Other Psychoactive Substances

(71) Applicant: Function Better Inc., New York, NY (US)

(72) Inventor: Alastair Greer, New York, NY (US)

(21) Appl. No.: 17/666,006

(22) Filed: Feb. 7, 2022

Related U.S. Application Data
(60) Provisional application No. 63/146,703, filed on Feb. 7, 2021.

(51) Int. Cl. A61K 31/05; G16H 20/10

(52) U.S. Cl. CPC A61K 31/05; G16H 20/10

(57) ABSTRACT

A method of treating a clinical condition in a subject with a psychoactive substance such as a cannabinoid (e.g. CBD or THC) or a psychedelic drug. The user is provided with a therapeutic product containing the psychoactive substance. For example, the therapeutic product may be one or more vials of CBD oil. The user interacts with an online program (e.g. via a smartphone app) that instructs the user about their dosing of the therapeutic product. Depending on the progress of their symptoms, the online program may instruct the user to increase their dose, reduce their dose, or keep the same dose. The online program continues for a set duration of time (e.g. nine days). As a result of completing the program, the user will have a more personalized dosing of the therapeutic product.

### TECHNICAL FIELD

This invention relates to the use of cannabinoids and other psychoactive substance.

### BACKGROUND

Although there has been great progress in the use of Cannabis medications for the treatment of mental and physical conditions, there are still problems with correct and accurate dosing. This is especially a problem because Cannabis medications are sold in many different forms with a wide range of potencies. Thus, there is much confusion about proper dosing and lack of means for tailoring doses to an individual's needs. Personalized dosing is important because there are many factors that determine a person's response to Cannabis medications. Such factors include the type of benefit being sought (e.g. pain, anxiety, etc.), severity of symptoms, which particular cannabinoid(s) are being used, body type and weight, and inherent variability in drug sensitivity and metabolism.

Moreover, there is buildup of tolerance that causes loss of marginal benefit from the increased dosing. As a result, many people use excessive doses of Cannabis medications—more than is needed to adequately treat their symptoms. Moreover, as the dose amount increases, the user may experience more unwanted psychoactive effects. This is especially true in the case of cannabidiol (CBD) products, which are intended to eliminate or minimize any psychoactive effects. The successful use of Cannabis for medication purposes depends much on managing its intoxicating or cognition impairing properties. There needs to be a more effective way to achieve a dosage that is personalized and best suited for that individual user. For long-term use of Cannabis medications, it is important to find the optimum personalized dosing. Likewise, as other psychedelic substances (e.g. ketamine, LSD, psilocybin, etc.) are gaining more acceptance, there is a need for a more effective way of finding the optimum dose of such psychedelic substances for the user.

### SUMMARY

This invention is a method of treating a clinical condition in a subject with a psychoactive substance. This invention titrates a dose amount of the psychoactive substance based on symptoms reported by the user. Psychoactive Substance: Examples of psychoactive substances that could be used in this invention include cannabinoids (as explained further below) and psychedelics. Examples of psychedelics include ketamine, mescaline, psilocybin, LSD (lysergic acid diethylamide), DMT (N,N-Dimethyltryptamine), MDMA (3,4-ethylenedioxy methamphetamine), 2C-B (4-bromo-2,5-dimethoxyphenethylamine), 2C-I (iodo-2,5-dimethoxyphenylethan-1-amine), 5-MeO-DMT (5-methoxy-N,N-dimethyltryptamine), AMT (α-methyltryptamine), and DOM (2,5-dimethoxy-4-methylamphetamine).

Formulation: The psychoactive substance is provided as a therapeutic product in any suitable formulation for consumption by the user. Examples of orally-administered formulations of the psychoactive substance include oils, aqueous solutions, and edible food (e.g. gummy bears). In some embodiments, the psychoactive substance is provided in liquid form (e.g. oil or aqueous solution). The psychoactive substance may be provided from any suitable source, such as natural plant or organic material in raw or bulk form (e.g. mushrooms or plant material), extracts from natural source materials, chemical synthesis, partial chemical synthesis, etc. The psychoactive substance may be provided in any suitable container, such as dropper vials, syringes, etc.

Native product material may be supplied in natural, organic, whole, raw, native, crude, unprocessed, or minimally processed forms. In such cases, the precise amount of the relevant psychoactive substance may be unknown or the amount is only estimated. As such, for native product materials encompassed by this invention, the dose amount may be stated in terms of the bulk amount of the material, regardless of the exact chemical composition (e.g. mixture of phytochemicals) of the material. As an example, for psilocybin mushrooms containing psilocybin as the psychoactive substance, the dose amount may be expressed as the amount (e.g. grams) of the mushroom substance instead of the amount of the psilocybin compound itself. Expressing the dose amount in this alternate form may be useful for therapeutic materials that do not have a well-defined or consistent ingredient composition for the psychoactive substance.

In some embodiments, the invention provides a kit containing multiple different (two or more) therapeutic products for the same psychoactive substance. Each of the different therapeutic products contains a different concentration amount of the psychoactive substance. As used herein, the term ‘kit’ means that the products are packaged together in the same package.

Automated Dosing Protocol: The user is engaged with an automated dosing protocol that receives input from the user and sends instructions to the user about how much of the psychoactive substance to administer. These messaging exchanges may occur on any suitable regular basis, such as daily or weekly. The instructions for incremental increase in the dose amount could be given in volume parameters (e.g. ml or cc). In some embodiments, the dosing protocol is a predetermined dose escalation schedule (i.e. structured protocol).

The dosing protocol is performed over a duration of multiple days. In some embodiments, the days of the protocol are in consecutive sequence (days 1, 2, 3, 4, and so on). As used herein, ‘initial day’ means that day on which the protocol is started and ‘initial symptom score’ means the symptom score for that day. As used herein, ‘subsequent day’ means any day following the initial day (e.g. the second or third day of the protocol) and ‘subsequent symptom score’ means the symptom score for that day. As used herein, ‘later day’ means any day following the subsequent day (e.g. the third or fourth day of the protocol) and ‘later symptom score’ means the symptom score for that day.

### BRIEF DESCRIPTION OF THE DRAWINGS

FIGS. 1A and 1B show an example of a dosing protocol in flowchart format.

FIG. 2 shows another example of a dosing protocol in flowchart format.

FIG. 3 shows an example of a vial containing the therapeutic product and a medicine dropper with volume markings.

FIG. 4 shows the results of the experimental trial (in bar graph form) in subjects being treated for anxiety.

FIG. 5 shows the results of the experimental trial (in bar graph form) in subjects being treated for pain.

FIG. 6 shows the results of the experimental trial (in bar graph form) in subjects being treated for sleep/insomnia.

FIG. 7 shows the mean daily scores in bar graph form.

FIG. 8 shows the mean daily scores as a trend line.

### DETAILED DESCRIPTION OF EXAMPLE EMBODIMENTS

The following are specific example embodiments to assist in understanding the invention. This invention uses a psychoactive substance, which is provided as a therapeutic product. There are a variety of different types of psychoactive substances that could be used for this invention, as explained above. Cannabinoids: An example of a psychoactive substance that could be used in this invention is cannabinoids. As used herein, ‘cannabinoids’ means the phytochemical compounds found in Cannabis plants that have some kind of pharmacologic effect in humans. Examples of cannabinoids include tetra-hydro-cannabinol (THC), cannabidiol (CBD), cannabinol (CBN), and cannabigerol (CBG). The cannabinoid product may contain multiple different types of cannabinoid compounds. For example, ‘full spectrum CBD’ products contain extracts that include CBD and other cannabinoids in various amounts, including THC. Examples of CBD-dominant products have a greater proportion of CBD, but only a small amount of THC (for example, less than 0.3%).

The amount of CBD relative to THC is often considered an important feature that distinguishes one cannabinoid formulation from another. This THC/CBD balance is important to the physiologic effects experienced by the user (e.g. both therapeutic effects and unwanted side-effects). The cannabinoid product used in this invention may have a unique code or batch number, which is associated with a particular cannabinoid compound profile (i.e. amounts of each cannabinoid ingredient), as well as differences in the amount of terpenes. Thus, that particular cannabinoid ingredient profile can be matched with the specific dosing protocol associated therewith. That is, a particular cannabinoid product formulation should be matched with a particular dosing protocol that is specially designed therefor. This is especially important because the cannabinoids are extracted from naturally-grown plant material (as opposed to chemically synthesized), and therefore, cannabinoid ingredient profiles may vary from batch-to-batch. Moreover, the cannabinoid ingredient profile may vary across different product lines.

The cannabinoid product may further contain other nutritional supplement-type ingredients, such as magnesium, L-theanine, GABA (gamma-aminobutyric acid), adaptogens, or entheogens. Different formulations may be specially designed for particular treatments. For example, because CBN has sedative effects, a formulation of 180 mg CBD and 20 mg CBN (in 5 mL volume), plus magnesium, L-theanine, and GABA can be very effective for treating insomnia.

CBD-containing or CBD-dominant cannabinoid formulations are of particular interest for their therapeutic benefits. Examples of CBD-dominant products include those having CBD amounts at 10 mg/mL, 20 mg/mL, 40 mg/mL, 60 mg/mL, 80 mg/mL, etc. The doses derived therefrom may be expressed in multifold format, such as 1×, 2×, 3×, 4×, and so on. This may be provided in any bulk liquid volume, such as 5 mL, 10 mL, 15 mL, 30 mL, or 60 mL volume. These may contain total amounts of CBD in the range of 50-3,000 mg in any suitable combination with the bulk volume. The optimum selection of the product will depend on the dose amount that the user intends to consume.

To facilitate dose adjustments or to widen the range of dosing amount, the user could be supplied with kits containing two or more different cannabinoid products, each containing a different formulation of cannabinoid(s). For example, the user may have two CBD tincture bottles, with one that is 1×concentration and a second that is 2×concentration. Examples of this include the following two 15 mL bottle kits: (a) 100 mg and 200 mg to give a dosing range from 5-30 mg; (b) 200 mg and 400 mg to give a dosing range from 10-60 mg; (c) 50 mg and 100 mg to give a dosing range from 2.5-15 mg.

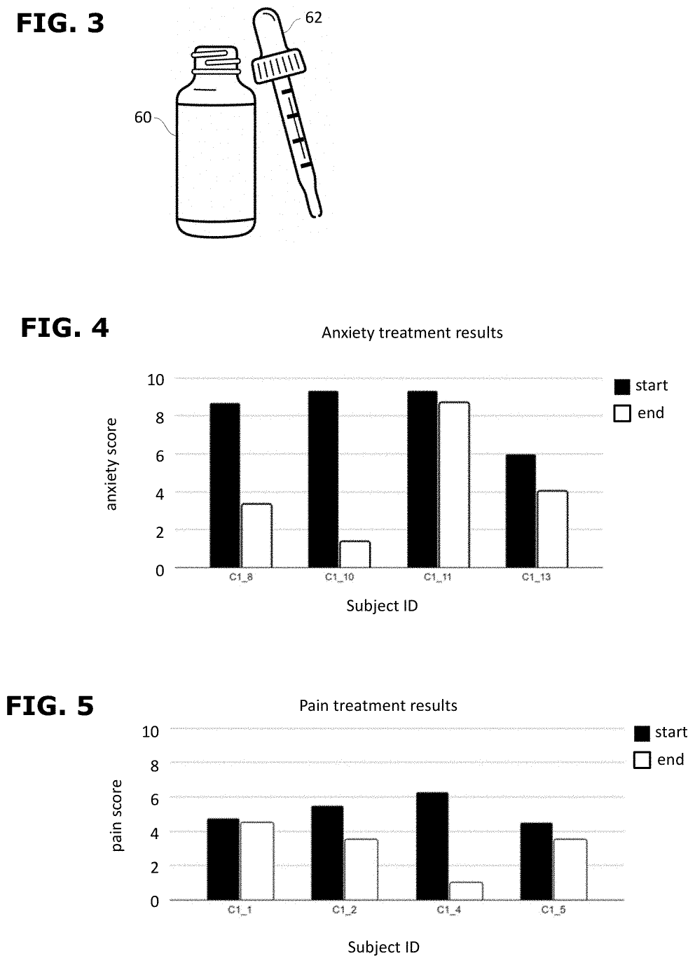

As an example, FIG. 3 shows a vial 60 containing CBD oil along with a graduated dropper 62 having liquid level markings for 0.25, 0.5, 0.75, and 1.0 mL. This allows the dosage instructions to be given in liquid volume. For example, “take 0.5 mL twice daily.” This invention could be applied to any suitable delivery forms for cannabinoids, including liquid formulations which are administered by mouth, such as oils, distillates, alcohol extracts, etc. Examples of other forms that could be used include oral sublingual or mucosal strips, oral sublingual or mucosal sprays, cartridges for use in precision-dosing vaporizers, gel tablets or capsules, etc.

Conditions & Symptom Scoring: This invention could be targeted for any clinical condition that can be improved by treatment with the psychoactive substance. Examples of such conditions include pain, sleep/insomnia, anxiety, stress, opioid and other addictions, alcoholism, mood depression, post-traumatic stress disorder, obsessive compulsive disorder, cancer, HIV/AIDS, amyotrophic lateral sclerosis (ALS), Parkinson's disease, multiple sclerosis, spinal cord injury with spasticity, epilepsy, inflammatory bowel disease, neuropathy, Huntington's disease, cachexia or wasting syndrome, nausea, and muscle spasms.

For the dosing protocol, the user chooses a particular condition to treat and makes a self-assessment of their condition by a standardized numeric scoring scheme. The scoring scheme can have any numeric scale and be adjusted according to factors that depend on the specific condition being evaluated. The scoring scheme may be in forward or reverse direction (e.g. higher scores representing worse severity, or alternatively, lower severity). One example of a scoring scheme is the ‘GAD-7 anxiety scoring’ questionnaire which rates anxiety based on seven categories and gives a composite numerical score of 0-21. In this scoring scheme, 0-4 indicates minimal anxiety, 5-9 indicates mild anxiety, 10-14 indicates moderate anxiety, and 15-21 indicates severe anxiety.

Another example of a scoring scheme is the commonly used ‘numeric pain rating scale’ that uses a scale of 0 (no pain) to 10 (worst pain imaginable) for measuring pain. In this scoring scheme, 0 indicates no pain, 1-3 indicates mild pain, 4-6 indicates moderate pain, and 7-10 indicates severe pain. Another example is the ‘Jenkins sleep questionnaire’ which rates sleep quality on four items: (a) difficulty falling asleep, (b) waking up several times per night, (c) difficulty staying asleep (including waking up too early), and (d) waking up feeling tired and worn out after usual amount of sleep. A score rating of 1-6 is given based on the number of nights these problems are experienced over the previous four weeks.

User Messaging: In this invention, messages are sent to a user's computing device, such as a mobile computer device. Examples of such include tablet computers, laptop computers, handheld computers, personal digital assistants, smartphones, or other types of personal mobile devices. The mobile computer device is equipped with wireless network communication capabilities (e.g. for communicating on Wi-Fi or cellular networks). Any suitable automated and personalized communication platform could be used, including services or applications that communicate with the user via text messages, emails, phone calls, video calls, intelligent chatbots, notifications on a stand-alone app, etc. This platform could also be used for communication and sharing of data with healthcare professionals. An example of how the messaging system could be configured is described in U.S. Pat. No. 11,135,367 (Andrew Tubb), which is incorporated by reference herein.

Dose Increase Titration: In one embodiment, this invention provides a protocol for gradually increasing a user's dosage (i.e. dose titration). The user selects a condition to be treated with the psychoactive substance. The user may be asked useful personal and health information such as name, email, phone number, gender, age, weight, height, etc. To begin the protocol, the user is asked to select a clinical condition for which treatment is desired. The user self-assesses their condition and determines their symptom score using the relevant standardized scoring scheme. The user reports their initial symptom score. The protocol instructs the user to begin with an initial dose amount of the therapeutic product.

On each following day, the user reports their symptom score. The symptom score may be the same, improved, or worsened. Depending on the change (if any) in the symptom score, the user is given options and instructions to best manage their dose titration. This may involve instructing them to stay at the same dose level, increase their dose level in an incremental manner, pausing the protocol, giving reassurance to the user, or informing them about self-care and wellness management to supplement their treatment, such as recommendations on food diet, exercise, and stress relief techniques.

### EXAMPLE 1

In this example This is an example of an automated text message exchange with a user for a 9 day (consecutive) dose titration protocol is sufficient to achieve a minimum effective dose. In this example, Twilio was the automated text messaging service that was used. The cannabinoid medication kit is three 15 ml bottles of CBD tincture (i.e. CBD mixed into an ethyl alcohol base): bottle #1, bottle #2, and bottle #3. Bottle #1 contains CBD at a concentration of 100 mg in 5 ml volume (20 mg/ml). Bottle #2 contains CBD at a higher concentration (2×) of 200 mg in 5 ml volume (40 mg/ml). Bottle #3 contains CBD at a further higher concentration (3×) of 300 mg in 5 ml volume (60 mg/ml). Because the three bottles have different dose amounts, to facilitate identifying the correct bottle and avoid mistakes, bottle #1 has a blue-colored label, bottle #2 has a yellow-colored label, and bottle #3 has an orange-colored label. The user inputs the product code or batch number. This ensures that the relevant dosing protocol is associated with the correct cannabinoid product. The user indicates that the therapeutic goal is treating pain, i.e. reducing pain.

On day #1, the user reports a pain score of 8, indicating severe pain. Accordingly, the user is instructed to take the first dose of 6.25 mg from the (blue bottle) twice daily, and continue this dose regimen for the next three days. To facilitate correct adherence to the instructions, the user is shown an image of the blue bottle and the correct liquid level marking on the graduated dropper. On day #2, the user reports a pain score of 6, indicating a reduction in pain symptoms to a moderate level. This indicates that the CBD treatment is working and accordingly, the user is instructed to continue the same dosing regimen (i.e. 6.25 mg twice daily). On day #3, the user reports a pain score of 6, indicating that the pain level remains the same. This indicates that the CBD treatment is suppressing the pain and accordingly, the user is instructed to continue the same dosing regimen (i.e. 6.25 mg twice daily).

On day #4, the user reports a pain score of 5, indicating a reduction in pain symptoms. This indicates that the CBD treatment is continuing to be effective and accordingly, the user is instructed to switch to the yellow bottle (40 mg/ml) and take 12.5 mg twice daily. The user is shown an image of the yellow bottle and the correct liquid level marking on the graduated dropper. On day #5, the user reports a pain score of 7, indicating a flare-up of the pain symptoms. The user is asked whether there were any external factors that might have caused this flare-up. Here, the user replies “no”, indicating that the CBD treatment might be ineffective for the user. Thus, the user is asked whether to pause the protocol to allow the user to reassess whether this treatment will be beneficial. Here, the user desires to continue CBD treatment.

Accordingly, the user is given reassurance and instructed to continue at the same dose (i.e. 12.5 mg twice daily). On day #6, the user reports a pain score of 5, indicating that the pain flare-up has resolved. This indicates that the CBD treatment is continuing to work and accordingly, the user is instructed to continue the same dosing regimen (i.e. 12.5 mg twice daily).

On day #7, the user reports a pain score of 4, indicating that the CBD treatment is continuing to be effective. It is now time for the user to increase the dose amount to stabilize the pain at that level. Accordingly, the user is instructed to switch to the orange bottle (60 mg/ml) and take 18.75 mg twice daily. The user is shown an image of the orange bottle and the correct liquid level marking on the graduated dropper. On day #8, the user reports a pain score of 4, indicating that the pain level is stable. Accordingly, the user is instructed to continue the same dosing regimen (i.e. 18.75 mg twice daily). On day #9, the user reports a pain score of 4, indicating that the pain level continues to be stable. Accordingly, the user has reached the personalized minimum effective dose and is instructed to continue the same dosing regimen (i.e. 18.75 mg twice daily) from hereon. At the end of the protocol, the user also receives a summary report that indicates the minimum effective dose, the lowest pain score achieved, the starting and ending benchmarks, and a graph plotting the daily symptom scores.

### EXAMPLE 2

Another example of an ‘up dose’ titration for the treatment of pain is as follows. This interaction is based on a score system of 0-10 with greater numeric values indicating worse pain (none, mild, moderate, severe). If the score is improved, then the user is given an option to either remain at the same dose level or increase their dose level. The user may or may not be given a recommendation on which option to elect. If the user selects the option to increase their dose level, then the user is instructed to increase the dose by an amount X.

If the user's reported score is the same as before, then the user is told to increase the dosage by an incremental amount X, where X represents an amount that is in the range of 50-300% increase relative to the current dose. This dosage increase may involve switching or combining the cannabinoid products. The increased amount X will depend on the particular cannabinoid product being titrated. In the case of CBD, the amount X could be 2.5-25 mg added to the current dose. Alternatively, the amount may be given as a larger volume of the product that is equivalent to the dose increase (e.g. increase by 0.25 mL).

If the score is the same, then the user is given a recommendation to increase their dose level. The user is given the option to do so. If the user chooses an increase in dose, then the user is instructed to increase to X amount. The amount may be given as volume (e.g. increase by 0.25 mL) or weight (e.g. milligrams). If the score worsens (i.e. increases), then the user is asked if they are experiencing any transient external factors that might have contributed to the deterioration. This is to explore the possibility that their decline in status may be a transient setback. If the user answers affirmatively about the external factors, then the user is reminded about routine self-care and wellness management to supplement their cannabinoid treatment, such as recommendations on food diet, exercise, and stress relief techniques. The user is also asked whether they wish to increase their dose amount. If the user chooses to increase their dose, then the user is instructed to increase their dose by an amount X, as explained above.

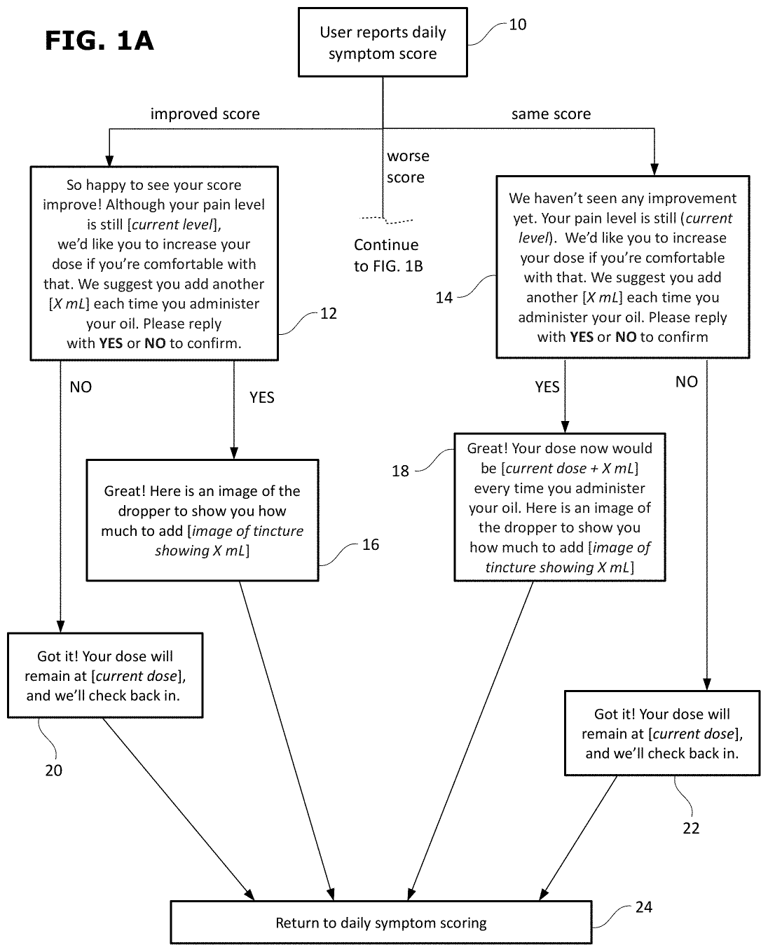

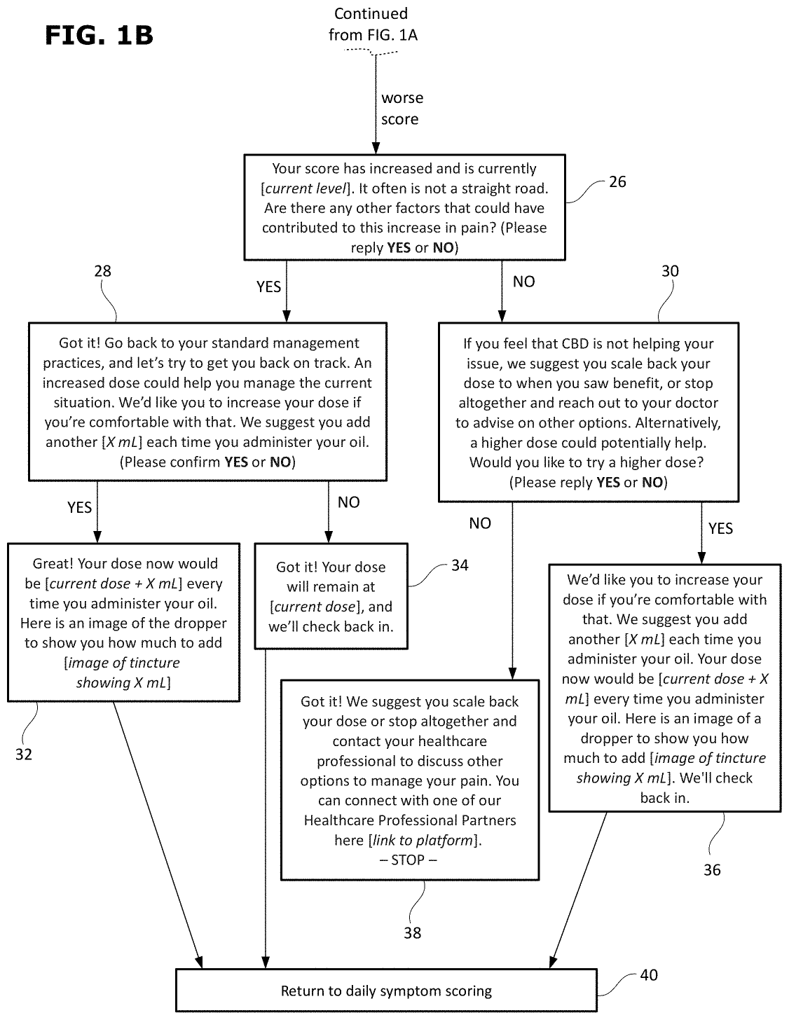

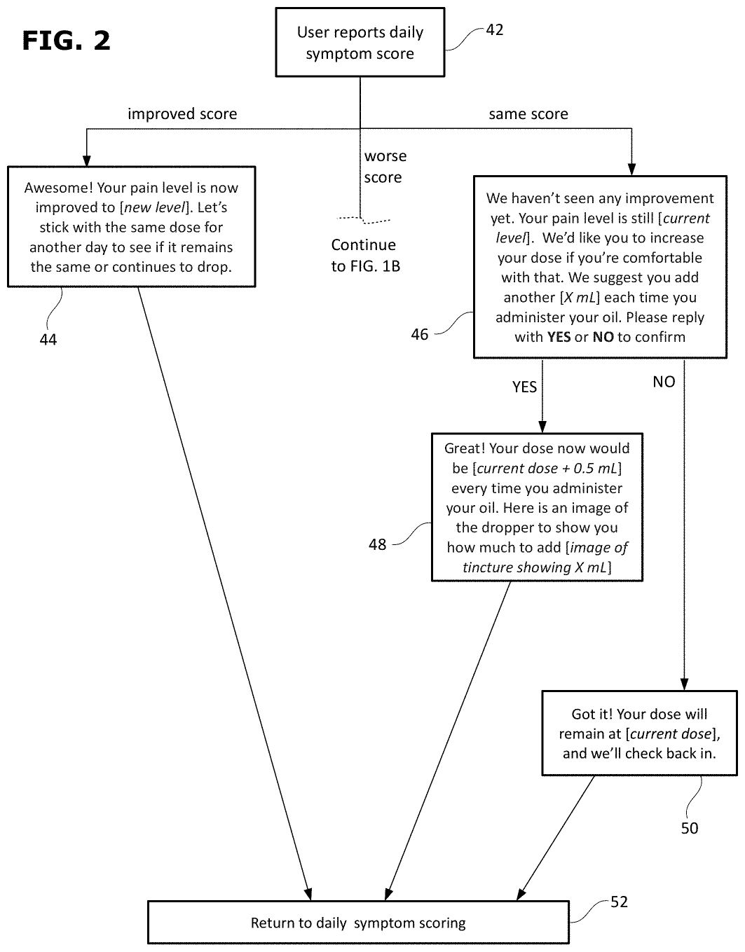

If the user chooses to not increase their dose, then they are advised to reduce their dosage or discontinue their cannabinoid treatment. They may further be advised to consult with a health care professional to discuss other therapeutic options. FIGS. 1A and 1B show an example of a message exchange in flowchart format. Flowchart box 10 is the user report of their daily symptom score. If the score is improved compared to prior score(s), then the user is given the message shown in box 12. The user is given a choice of increasing their dose amount. Answering ‘yes’ gives the message shown in box 16. Answering ‘no’ gives the message shown in box 20. As an iterative process, box 24 shows that the dosing procedure returns back to the reporting of the daily symptom score (box 10).

Turning back to the alternate pathway from box 10, if the symptom score is the same, then the user is given the message shown in box 14. The user is given a choice of increasing their dose amount. Answering ‘yes’ gives the message shown in box 18. Answering ‘no’ gives the message shown in box 22. As an iterative process, box 24 shows that the dosing procedure returns back to the reporting of the daily symptom score (box 10).

Turning back to yet another alternate pathway from box 10, if the symptom score is worse, then the user is given the message shown in box 26. See FIG. 1B, which shows a more complex dose escalation procedure. The user is asked whether other transient factors could have caused the worsening symptoms. Answering ‘yes’ gives the message shown in box 28. From there, the user is given a choice of increasing their dose amount. Answering ‘yes’ gives the message shown in box 32. Answering ‘no’ gives the message shown in box 34. As an iterative process, box 40 shows that the dosing procedure returns back to the reporting of the daily symptom score (box 10).

Returning to the alternate pathway from box 26, if the user answers ‘no’ to the question, the user is given the message shown in box 30 suggesting a number of options that the user could select. The user is further asked whether they wish to increase their dose amount. Answering ‘yes’ gives the message shown in box 36. As an iterative process, box 40 shows that the dosing procedure returns back to the reporting of the daily symptom score (box 10). Answering ‘no’ gives the message shown in box 38, which instructs the user to discontinue the treatment or reduce their dosage, and recommends consultation with a healthcare professional.

FIG. 2 shows another example of a message exchange for a dosing protocol in flowchart format. Flowchart box 42 is the user report of their daily symptom score. If the score is improved compared to prior score(s), the user is given the message shown in box 44, which instructs the user to continue at the same dosage amount. If the symptom score is the same, then the user is given the message shown in box 46. The user is given a choice of increasing their dose amount. Answering ‘yes’ gives the message shown in box 48. Answering ‘no’ gives the message shown in box 50. As an interactive process, box 52 shows that the dosing procedure returns back to the reporting of the daily symptom score (box 42).

Protocol Duration: In some embodiments, the up-dose protocol is performed for a duration of about 9 days (i.e. 9±1 days). This invention could also use other protocol durations instead of the 9 day duration explained above. These could be shorter duration dose titrations. In some embodiments, the protocol is performed over a duration of 3 days. In some embodiments, the protocol is performed over a duration of 6 days. These shorter-length protocols may be effective for those who have already had an initial full-duration titration (e.g. the 9 day protocol) and wish to do fine-tuning of their dosing. These could also be useful for users having more severe symptoms and needing a faster ramp-up in dosing. In some embodiments, the protocol is performed over a duration of 7-15 days. In some embodiments, the protocol is of 6 day duration.

Dose Reduction Titration: In some embodiments, this invention gives the user the opportunity to try a ‘dose reduction titration’ to further fine-tune their personalized dose amount. Various parts of this ‘down-dose’ titration may have similar features as above, except that the goal is to titrate down to a reduced dose amount. The following is an example down-dose titration in which the duration is 3 days.

The user begins by self-assessment of their condition and determining their symptom score. The user reports their initial symptom score and continues to report the scores during the 3 day duration. Depending on whether the reported symptom score changes or remains stable, the user is instructed to reduce the dosage amount. For example, the user could be instructed to reduce the CBD dose amount by 1-5 mg for each twice daily dosing. This may be achieved by the user drawing out a smaller volume from the same bottle using the graduated dropper. Or alternatively, the user may be instructed to switch to a lower dose concentration bottle (e.g. back to the yellow 40 mg/ml). This scoring and dose reduction is performed daily for 3 days duration to reach a lower personalized minimum effective dose than the starting dose.

The down-dosing may be contiguous with the up-dosing or be separated by a duration of time of at least one day (e.g. 3 days interval). For example, this could be a 12-day duration titration with an initial 9-day up-dose titration followed immediately by a 3-day down-dose titration to fine tune the minimum effective dose.

Drug Holiday: The user may develop tolerance to the psychoactive substance whereby the therapeutic efficacy declines. As such, the purpose of a ‘drug holiday’ is to temporarily discontinue treatment for a duration so that the user becomes re-sensitized to the psychoactive substance. The user may self-initiate a drug holiday or it may be suggested to the user by a message sent to the user. In a drug holiday protocol, the user is instructed to temporarily interrupt the treatment in a structured manner. The user may also receive messages informing them about routine self-care and wellness management, such as recommendations on food diet, exercise, and stress relief techniques.

The drug holiday is intended to be only temporary. In some embodiments, the drug holiday protocol is for a duration of one week or shorter, and in some cases, for a duration of 2-3 days. The drug holiday protocol is followed by the up-dose protocol described above. After completion of this drug holiday and up-dose cycle, the user may be at a lower daily dose than prior to the drug holiday.

Miscellaneous: The various protocols described herein could be used in combination or repeated in cycles. One or more of the protocols could be repeated intermittently. Also, the user could be actively engaged by automated messaging to try the different protocols. Data Collection: Data from multiple users may be collected and stored for study purposes (e.g. study clinical efficacy). The user may be asked to allow sharing of their information. Such data could be de-identified (would have the patient data, but stripped of identifying information) for data sharing with healthcare organizations. User Cohorts: By curating and analyzing the data collected from users, as standardized according to the therapeutic product formulation, the delivery method and user outcome could be organized by cohorts according to the user profiles. By having this organized cohort data, a user could be matched with a cohort profile and given a recommendation on which therapeutic product to use based on the outcome data for that cohort profile.

### Experimental Trial

An experimental trial was conducted on 11 subjects who followed a 9-day dosing protocol for CBD oil. The subjects sought treatment for anxiety, pain, or sleep. On a daily basis, the subjects rated the intensity of their symptoms on a 0-10 numeric scale, with 0 being best and 10 being worst intensity. This scale was constructed similar to the standard ‘Numeric Pain Rating Scale’ often used in clinical practice for pain assessment.

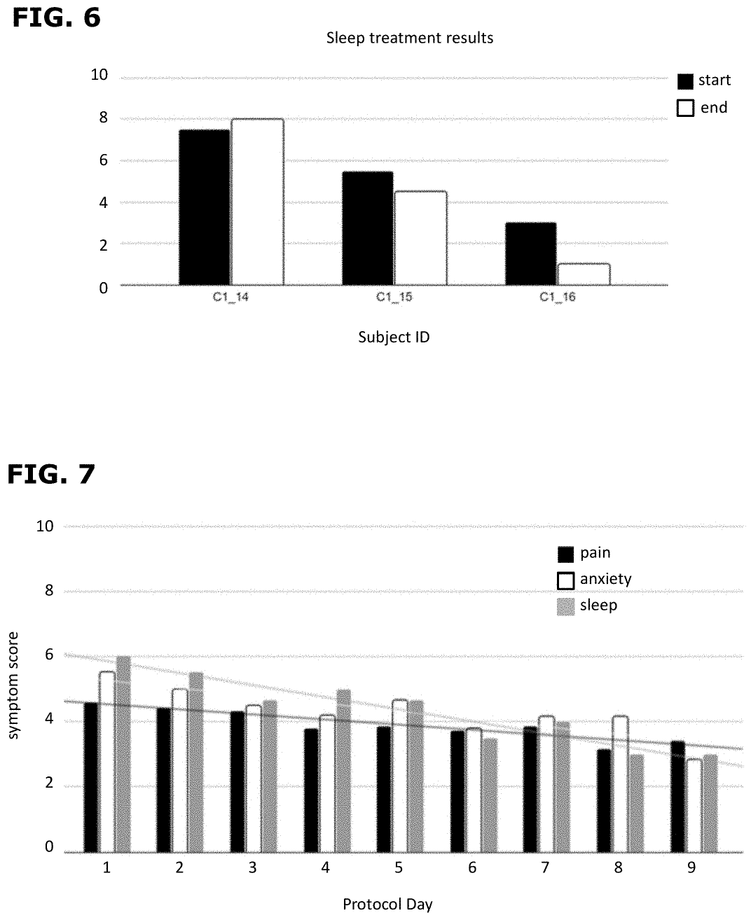

The results are shown in FIGS. 4-6. FIG. 4 shows a bar graph for four subjects being treated for anxiety. As seen here, all subjects improved (comparing the initial and end scores) after completion of the protocol. FIG. 5 shows a bar graph for four subjects being treated for pain. As seen here, all subjects improved (comparing the initial and end scores) after completion of the protocol. FIG. 6 shows a bar graph for three subjects being treated for sleep/insomnia. As seen here, all subjects improved (comparing the initial and end scores) after completion of the protocol.

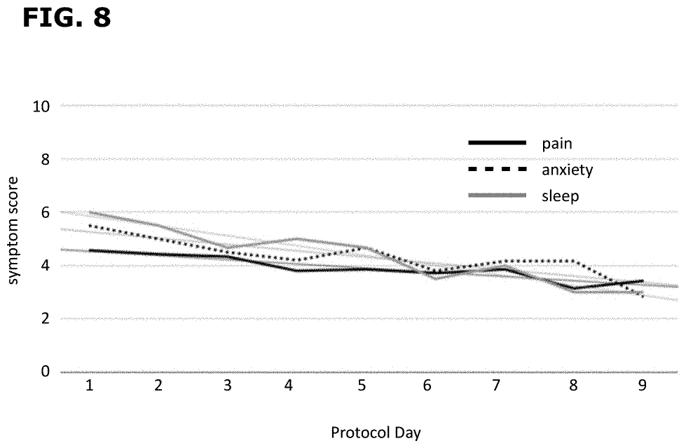

The trial further enrolled five additional subjects. The results for all 16 subjects combined and averaged (mean scores) are shown in FIGS. 7 and 8. FIG. 7 shows a bar graph of the mean scores for the subjects being treated for either anxiety, sleep, or pain. Days 1-3 were a maximum of 5 mg CBD; days 4-6 were a maximum of 10 mg CBD; days 7-9 were a maximum of 20 mg CBD. This shows the mean daily score of all 16 subjects, for their respective symptoms.

As seen here, the mean scores improved after completion of the protocol (comparing the initial and end scores). FIG. 8 shows the same data with trend lines (instead of bar graph). This shows that the mean daily scores exhibit a downward trending line (i.e. gradual symptom improvement).

Representative Subject #C1_10 was a 25 year-old female seeking treatment for anxiety. She started with an anxiety score of 7, and improved to a lower score of 2 after completion of the protocol. Her minimum optimal dose was 10 mg of CBD/day. Representative Subject #C1_4 was a 46 year-old male seeking treatment for pain. He started with a pain score of 3, and improved to a lower score of 0 after completion of the protocol. His minimum optimal dose was 10 mg of CBD/day. Representative Subject #C1_8 was a 31 year-old female seeking treatment for anxiety. She started with an anxiety score of 5, and improved to a lower score of 1 after completion of the protocol. Her minimum optimal dose was 20 mg of CBD/day.

The descriptions and examples given herein are intended merely to illustrate the invention and are not intended to be limiting. Each of the disclosed aspects and embodiments of the invention may be considered individually or in combination with other aspects, embodiments, and variations of the invention. In addition, unless otherwise specified, the steps of the methods of the invention are not confined to any particular order of performance. Modifications of the disclosed embodiments incorporating the spirit and substance of the invention may occur to persons skilled in the art, and such modifications are within the scope of the invention.

Any use of the word “or” herein is intended to be inclusive and is equivalent to the expression “and/or,” unless the context clearly dictates otherwise. As such, for example, the expression “A or B” means A, or B, or both A and B. Similarly, for example, the expression “A, B, or C” means A, or B, or C, or any combination thereof.

1. A method of treating a clinical condition in a subject, comprising:
having a therapeutic product comprising a psychoactive substance at a predetermined dose concentration;
performing a dosing protocol that comprises the steps of:
on an initial day, providing input about the clinical condition to an automated dosing protocol program to determine an initial symptom score;
administering a starting dose amount of the therapeutic product;
on a subsequent day, providing input about the clinical condition to the automated dosing protocol program to determine a subsequent symptom score;
based on a comparison between the subsequent symptom score and the initial symptom score, sending an electronic message to the subject giving instructions to increase the dose amount;
administering an increased dose amount of the therapeutic product.

2. The method of claim 1, further comprising:
on a later day, providing input about the clinical condition to the automated dosing protocol program and receiving a later symptom score;
based on a comparison between the later symptom score and the subsequent or initial symptom score, sending an electronic message to the subject giving instructions to further increase the dose amount.

3. The method of claim 1, further comprising, based on the comparison between the subsequent symptom score and the initial symptom score, sending an electronic message to the subject giving instructions to maintain the same dose amount.

4. The method of claim 2, further comprising, based on the comparison between the later symptom score and the subsequent or initial symptom score, sending an electronic message to the subject giving instructions to maintain the same dose amount.

5. The method of claim 1, wherein the increase in the dose amount is in the range of 40-250%.

6. The method of claim 2, wherein the further increase in the dose amount is in the range of 40-250%.

7. The method of claim 1, wherein the therapeutic product is in liquid form, and the method further comprises having a medicine dropper with volume markings and wherein the instructions for the dose amounts are given in volume parameters.

8. The method of claim 7, wherein the increase in dose amount is in an increment of about 0.25 or about 0.5 mL.

9. The method of claim 1, wherein the psychoactive substance is a psychedelic.

10. The method of claim 1, wherein the psychoactive substance is a cannabinoid.

11. The method of claim 10, wherein the increase in dose amount is in the range of 3.5-50 mg of the cannabinoid.

12. The method of claim 1, wherein the dosing protocol is performed over a duration of 7 to 15 days.

13. The method of claim 1, wherein the therapeutic product is associated with a batch identifier; and wherein the dosing protocol is matched to the batch identifier.

14. The method of claim 13, wherein the therapeutic product is selected from a selection of multiple different therapeutic products containing different amounts of the psychoactive substance, wherein the multiple different therapeutic products are each associated with a different batch identifier.

15. The method of claim 1, further comprising having a kit that comprises the first therapeutic product, wherein the dose concentration of the psychoactive substance is a first dose concentration; and
wherein the kit further comprises a second therapeutic product comprising the psychoactive substance at a second dose concentration, wherein the second dose concentration is greater than the first dose concentration.

16. The method of claim 15, wherein the second dose concentration is 1.5-3.0 times more than the first dose concentration.

17. The method of claim 15, wherein the electronic message to the subject giving instructions to increase the dose amount includes instructions to switch from using the first therapeutic product to the second therapeutic product.

18. The method of claim 15, wherein the first therapeutic product is labeled with a different color than the second therapeutic product.

19. The method of claim 16, wherein the electronic message to the subject comprises:
an image of the first therapeutic product or its label; or
an image of the second therapeutic product or its label.

20. The method of claim 15, wherein the psychoactive substance is a cannabinoid.
